(19) United States
(12) Patent Application Publication
(10) Pub. No.: US 2019/0248848 A1
(43) Pub. Date: Aug. 15, 2019

(54) SYNTHETIC TRANSCRIPTIONAL REGULATOR COMPOSITIONS AND METHODS

(71) Applicant: Arizona Board of Regents On Behalf of Arizona State University, Scottsdale, AZ (US)

(72) Inventors: Karmella Haynes, Phoenix, AZ (US); Stefan Tekel, Tempe, AZ (US)

(21) Appl. No.: 16/274,770

(22) Filed: Feb. 13, 2019

Related U.S. Application Data
(60) Provisional application No. 62/630,352, filed on Feb. 14, 2018.

(51) Int. Cl. C07K 14/47; C07K 14/00; C12N 15/85

(52) U.S. Cl. CPC C07K 14/47; C07K 14/001; C12N 15/85; C07K 2319/90; C07K 2319/35; C07K 2319/71; C07K 2319/80; C07K 2319/09

(57) ABSTRACT

Compositions and methods relating to synthetic modification of histone-binding proteins. Some histone-binding fusion-protein compositions include a modified polycomb chromodomain (PCD) motif, for example two tandem copies of the H3K27me3-binding PCD at the N-terminus separated by a linker. Some methods relate to multivalent engagement of one or more histone proteins or to tuning the activity of a synthetic histone-binding transcriptional regulator, for example by contacting the one or more histone proteins with a histone-binding fusion-protein composition having a modified PCD motif.

### CROSS-REFERENCE TO RELATED APPLICATION

This application claims priority to U.S. Provisional Patent Application Ser. No. 62/630,352, filed on Feb. 14, 2018.

### STATEMENT REGARDING FEDERALLY SPONSORED RESEARCH

This invention was made with government support under K01 CA188164 awarded by the National Institutes of Health. The government has certain rights in the invention.

### BACKGROUND

The discovery of histone post-translational modifications (PTMs) and the peptides that specifically interact with these marks has enabled scientists and cell engineers to manipulate chromatin, the DNA-protein structure that regulates gene expression states in eukaryotic cells.

### SUMMARY OF THE DISCLOSURE

Embodiments disclosed herein relate to compositions and methods involving synthetic modification of histone-binding proteins.

In some embodiments, histone-binding fusion-protein compositions include a modified polycomb chromodomain (PCD) motif, for example two tandem copies of the H3K27me3-binding PCD at the N-terminus separated by a linker. Such linkers can range in size (number of amino acids) and composition, including flexible or rigid linkers.

In other embodiments, methods are disclosed that relate to multivalent engagement of one or more histone proteins by a transcription regulator protein, for example by contacting one or more histone proteins with a histone-binding fusion-protein composition having a modified PCD motif. In some embodiments, the modification comprises two tandem copies of the H3K27me3-binding PCD at the N-terminus.

In further embodiments, methods are disclosed that relate to tuning the activity of a synthetic histone-binding transcriptional regulator, for example, the transcription regulator protein may include a histone-binding fusion-protein with a modified polycomb chromodomain (PCD) motif.

Thus, embodiments herein relate to methods to engineer proteins that are designed to activate or repress the expression of genes in living cells, with an objective being to activate or repress target genes based on “epigenetic” states instead of their DNA sequences.

### BRIEF DESCRIPTION OF THE FIGURES

Advantages of embodiments of the present disclosure will be apparent from the following detailed description of the exemplary embodiments thereof, which description should be considered in conjunction with the accompanying drawings in which:

FIG. 1 depicts a 3D model layout to show the plausibility of Pc2TF binding to adjacent H3K27me3 marks. (A) PCD (CBX8) in complex with trimethyl lysine (PDB 3I91). Three residues form a hydrophobic cage and surround the Kme3 moiety (inset). (B) H3K27me3 recognition by synthetic fusion proteins that carry a single or tandem PCD domains (PcTF and Pc2TF, respectively). The 3D rendering was composed in the PyMOL Molecular Graphics System, Version 1.3 Schrödinger, LLC (www.pymol.org/) using data for CBX8/H3K9me3 (PDB 4X3K), and a whole nucleosome assembly (PDB 5AV8) from the Protein Data Bank.

FIG. 2 shows a comparison of Pc₂TF variants that were expressed in a bacterial cell-free expression system. (A) Map of the expression vector and open reading frames (ORFs). Fusion-encoding ORFs were cloned in the pET28 vector at BamHI and XhoI. (B) Real-time detection of mCherry fluorescence was used to determine expression of recombinant protein in TXTL in a 96-well PCR plate in a Roche thermal cycler. Each replicate is an independent TXTL reaction in a single well (1 replicate for Pc_{Δ}TF and TXTL without DNA, 3 replicates for others). Replicates were pooled for ELISA's in panel C. Solid line=mean, shaded regions=SDM. (C) The bar chart shows mean signal from anti-mCherry-HRP signal at an absorbance of 450 nm (3 ELISA wells) from TXTL-expressed fusion proteins or plasmid-free “blank” TXTL captured by tethered trimethyl-K27 (K27me3), unmodified (K27), or modified non-target (K27ac) histone H3 peptides. For each TXTL product, individual values are normalized to the unmodified H3 mean value within the set (error bars=SDM).

FIG. 3 depicts a bivalent PCD fusion peptide shows enhanced avidity for H3K27me3 in microspot array experiments. (A) For high-yield expression, E. coli was transformed with pET28 plasmids encoding Pc_{Δ}TF (negative control), PcTF (single PCD), and the Pc₂TF containing the flexible linker SEQ. ID. NO. 1(GGGGS)₄. Native polyacrylamide gel electrophoresis (PAGE) of overexpressed proteins purified from E. coli. (B) Test slides were spotted with histone H3 peptides (K27me3 or unmodified K27) as indicated in the grid for qualitative analysis. Pseudo-colored images show mCherry signal after an application of 1.0 μM of fusion protein to individual arrays. (C) New arrays were spotted with 10, 20, and 50 μM H3K27me3 for quantitative analysis. Fluorescence signal versus the concentration of fusion protein applied to the array was used to calculate the apparent dissociation constant (Ka_{d}^{app}, not applicable for Pc_{Δ}TF). Each point in the graph is the mean signal from four spots in one application (error bars=SDM). The data displayed in the graph are from representative applications (out of four total) for 20 μM immobilized H3K27me3.

FIG. 4 illustrates that bivalent Pc2TF shows cooperative and on-target binding with H3K27me3 ligands. (A) The scatter plot shows mean HRP signal at an absorbance of 450 nm (one ELISA trail, means of four technical replicate wells, error bars=SDM) from wells in which 0.1 μM purified protein (Pc2TF, PcTF, or PcΔTF) was allowed to bind with different proportions of H3K27me3 biotinylated peptides (0-100%) mixed with unmodified H3 and tethered to neutravidin-coated surfaces. (B) Hill curves were fit to data for three ELISA trials (dots=technical replicate wells from all trials). (C) ELISA was used to detect interaction of 0.05 μM purified proteins with immobilized histone H3 peptides that were trimethylated at lysine 27, 4, or 9 or unmodified. The bar chart shows mean signal values from anti-mCherry-HRP at an absorbance of 450 nm (4 technical replicates, bars=SDM).

FIG. 5 demonstrates that Pc2TF stimulates expression at a Polycomb-silenced reporter gene. (A) An engineered HEK293 cell line, Gal4-EED/luc, was used for doxycycline-mediated control of H3K27me3 and PRC-mediated silencing at a Tk-luciferase reporter. Expression is partially silenced prior to dox treatment, as demonstrated previously 40 and becomes fully repressed at 96 hours. (B) Fusion constructs were cloned into the MV10 vector at XbaI. Fluorescence microscopy confirmed nuclear localization of the fusion proteins in transfected cells. The same samples were used for Western blots to confirm cellular expression of full length proteins, RT-qPCR to measure mRNA levels (C), and flow cytometry to measure RFP signal (D). Changes in the expression states of Tk-luciferase in 96-hour dox-treated cells were determined by RT-qPCR (C) and luc activity assays (D) (circles=replicates, described in Methods). (E) Fusion proteins were expressed in cells treated with dox for 0, 48, or 96 hours to determine the activity of the fusion activators at intermediate repressed states (bars=mean values for 3 luciferase assays from one transfected sample, each scaled to mean PcΔTF luc/cell; error bars=SDM).

FIG. 6 schematically depicts an embodiment in action of a transcriptional regulator composition.

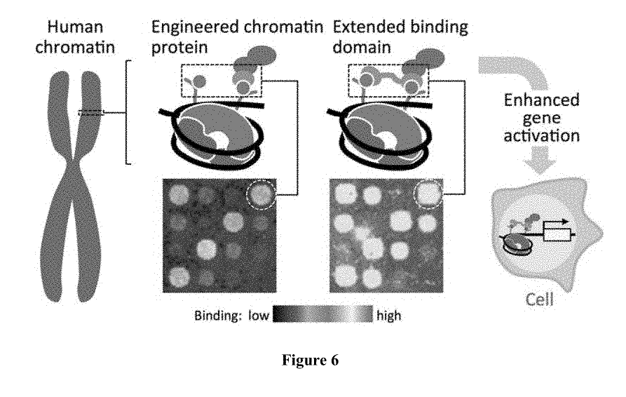

FIG. 7 shows predicted fluorescence after Pc-fusion binding and subsequent washing. A mathematical model was used to predict mCherry (RFP) signal (fluorophore concentration) when all H3K27me3 targets were saturated by PCD binding (t=0 sec) and over time during washing with buffer. A mechanistic model of washing of the spot assays was created using differential equations and solved using Matlab. Initial conditions were written to describe the initial amount of bound PcTF (1×10-6 M) and bound Pc2TF (0.5×10-6 M) to simulate saturated ligands—each Pc2TF is bound to two ligands, thus half as many Pc2TF molecules would saturate the surface. In one simulation, for washing a spot array with PcTF, an equation was written to describe PcTF association and dissociation with a single ligand as: dPb/dt=ka*Pu*Lu-kd*Pb. ka is the association rate constant, kd is the dissociation rate constant, Pu is the concentration of unbound PcTF or Pc2TF, Lu is the concentration of unbound ligand, and Pb is the complex of PcTF bound to ligand. Algebraic equations also track unbound PcTF (Pu=Ptotal−Pb) and unbound ligand (Lu=Ltotal−Pb). In the other simulation, for washing a spot array with Pc2TF, two equations were written. The first equation describes Pc2TF association with a ligand to form a complex bound by one PCD to one ligand (Pb 1) which can be lost either by dissociation or association of a second PCD or can be regained by one of the two PCDs of a Pb2 dissociating: dPb1/dt=ka*Pu*Lu-kd*Pb1 -ka*Vr*Pb1* Lu+kd*Pb2. These parameters are the same as above but also include Vr which is the ratio of overall concentration to effective local concentration (see Shewmake et al. 2008) and Pb2 which is a Pc2TF bound to two ligands via a PCD for each ligand. The second equation describes the second PCD of Pb1 binding to a ligand and dissociation of one PCD from a ligand: dPb2/dt=ka*Vr*Pb1*Lu-kd*Pb2. Algebraic equations also track unbound Pc2TF (Pu=Ptotal−(Pb1+Pb2)) and unbound ligand (Lu=Ltotal−1*Pb1−2*Pb2). Parameters were set to: ka=2000 M-1 s-1, kd=0.01 s-1, Ptotal=initial value of Pb (PcTF) or Pb2 (Pc2TF), Ltotal=1×10-6 M, Vr=100. Simulations can be run using most ordinary differential equation (ODE) solvers, but we used odel5s with absolute tolerance of 1e-9 and relative tolerance of 1e-6. Pb vs. time is plotted for PcTF. (Pb1+Pb2), Pb1, and Pb2 are plotted vs. time for Pc2TF. Matlab codes and equations used to generate these simulations can be found online: github.com/khaynes5/PcTF_kinetics.

FIG. 8 depicts additional peptide array binding data. Dot plots show representative results from microspot assay trials where varying concentrations of soluble fusion proteins were applied to 10, 20, or 20 uM of H3K27me3 (dots=means of 2 replicate spots, error bars=SDM). Apparent dissociation constant (Kdapp) values were determined by lines of best fit (dashed line). The table shows calculated Kdapp values, error (SDM), and R2 values for all experimental replicates (n/s=no signal, - - - =no additional replicate). Bold text indicates Kdapp values from curves that are shown in the representative dot plots.

### DETAILED DESCRIPTION

Aspects of this specification are disclosed in the following description and related drawings directed to specific embodiments. Alternate embodiments may be devised without departing from the spirit or the scope of the invention. Additionally, well-known elements of exemplary embodiments of the invention will not be described in detail or will be omitted so as not to obscure the relevant details. Further, to facilitate an understanding of the description, discussion of several terms used herein follows.

The word “exemplary” is used herein to mean “serving as an example, instance, or illustration.” Any embodiment described herein as “exemplary” is not necessarily to be construed as preferred or advantageous over other embodiments. Likewise, the terms “embodiments of the invention,” “embodiments” or “invention” do not require that all embodiments of the method, system or apparatus include the discussed feature, advantage or mode of operation.

The following abbreviations are used herein: ELISA, enzyme-linked immunosorbent assay; H3K27me3, histone H3 trimethylated at lysine 27; PCD, Polycomb chromodomain motif; PcTF, monovalent Polycomb-based transcription factor; Pc₂TF, bivalent Polycomb-based transcription factor; PTM, post translational modification.

It should also be understood that substantially similar components are at times identified using a single reference number for clarity. Further, it should also be understood that the present application makes reference to examples to aid in understanding features and advantages of the invention, and not to limit the invention to the examples herein.

In multicellular organisms, information relevant to cell state, tissue identity, and oncogenesis is often encoded as biochemical modifications of histones, which are bound to DNA in eukaryotic nuclei and regulate gene expression states. For example, “Polycomb-based transcription factor” (PcTF), a fusion protein that recognizes histone modifications through a protein-protein interaction between its polycomb chromodomain (PCD) motif and trimethylated lysine 27 of histone H3 (H3K27me3) at genomic sites, has been developed and validated. In another example, PcTF has been found to activate genes at methyl-histone-enriched loci in cancer-derived cell lines. However, PcTF induces modest activation of a methyl-histone associated reporter compared to a DNA-binding activator.

Therefore, in some embodiments, PcTF is modified to enhance its binding avidity. For example, we demonstrate the activity of a modified regulator called Pc₂TF, which has two tandem copies of the H3K27me3-binding PCD at the N-terminus. Pc₂TF has a smaller apparent dissociation constant value in vitro and shows enhanced gene activation in HEK293 cells compared to PcTF. Thus, in further embodiments, the intrinsic histone-binding activity of the PCD motif can be used to tune the activity of synthetic histone-binding transcriptional regulators.

In a specific embodiment, the Polycomb-based Transcription factor (PcTF) was constructed using a histone PTM-binding motif from the natural protein CBX8. The CBX8 effector protein binds to histone H3 trimethylated at lysine 27 (H3K27me3) through its N-terminal Polycomb chromodomain (PCD) and establishes a silenced transcriptional state. Expression of PcTF, an artificial transcriptional activator with an N-terminal PCD, mCherry tag, and C-terminal VP64 activation domain, led to increased expression of H3K27me3-enriched genes in three different cancer-derived cell lines.

These results show promise for designing transcription factors that can read chromatin marks to rewire aberrant epigenetic programming. However, binding affinities observed in vitro for isolated PCD domains is poor, reported as 5 to >500 μM, compared to DNA-binding domains with target affinities in the pico- to nanomolar range such as TALEs (˜3-220 nM), Zinc Fingers (˜0.01-16 nM), and CRISPR/Cas (˜0.5 nM). Stronger gene upregulation when mCherry-VP64 was targeted to a promoter via a Gal4 DNA binding domain compared to the PCD histone-binding domain also has been observed. PcTF-mediated gene activation is dose dependent and high PcTF expression levels are required for optimal activity. This limits the usefulness of PcTF for therapeutic applications where barriers to delivery severely limit the number of proteins that ultimately reach the nuclei of target cells. Although pharmacokinetic barriers to DNA and protein delivery in vivo are not trivial, increasing the effective dose of PcTF could significantly advance this technology towards clinical use.

The appearance of tandem histone binding domains within natural proteins suggests that the performance of histone-binding regulators can be customized and tuned through multivalency, defined as contact with more than one histone PTM via multiple domains. Multivalent chromatin proteins can engage adjacent PTMs within a single histone tail, such as K4me3 and R8me2 on histone H3 bound by Spindlinl, or K5ac and K12ac on histone H4 bound by TAF_{II}250. PTM targets can also reside on two distinct histone tails, such as H4K16ac and H3K4me3 bound by BPTF. Dual recognition of histone PTMs is accomplished by tandem protein motifs within the histone-binding protein. Comparisons of natural mono- and divalent proteins as well as histone peptide on- and off-target binding studies have produced compelling evidence that tandem motifs contribute to avidity and specificity.

The idea that combinatorial avidity allows proteins to read a “histone code” has been the topic of some controversy. Until recently, multivalency had not been demonstrated using a rationally designed composition of binding domains. Tandem histone binding domains have been used to design protein probes to fluorescently label regions that are enriched for specific histone modifications. To date, multivalency has not been used to design a transcriptional regulator and tandem PCDs have not been reported. In order to compensate for the modest affinity of the CBX8 PCD for its target, in some embodiments, we added a second copy of H3K27me3-binding PCD to the N-terminus of PcTF to build Pc₂TF. Here, we demonstrate that Pc₂TF shows stronger avidity for H3K27me3 in vitro. This activity corresponds with enhanced activation of a H3K27me3-repressed gene in cultured cells. These results have important implications for building and tuning fusion proteins that target sites of Polycomb-mediated silencing, which plays a central role in cancer and stem cell plasticity.

### Non-Limiting Examples

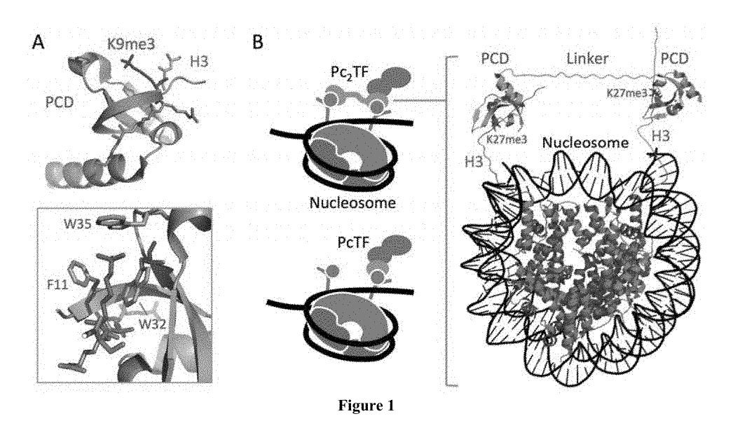

Design of a bivalent synthetic chromatin-based transcriptional regulator. We designed the Pc₂TF protein to simultaneously recognize two copies of the histone posttranslational modification H3K27me3. The Polycomb chromodomain motif (PCD) comprises three β strands packed against α C-terminal a helix, and a hydrophobic pocket formed by three aromatic residues that interact with a methyl-lysine sidechain (FIG. 1A). The arrangement of histones within the nucleosome octamer suggests that Pc₂TF might bind adjacent trimethylated H3K27 residues. A single nucleosome includes eight individual histone proteins. The central tetramer contains two copies of histone H3 and H4. The H3 proteins are oriented in cis so that the unfolded N-terminal tails protrude away from the nucleosome in the same direction (FIG. 1B). One or both H3 tails can become trimethylated at lysine 27 by the enzyme enhancer of zeste (EZH). Therefore, tandem PCDs in the multivalent protein Pc₂TF might interact with two histone post translational modifications (PTMs) in a single nucleosome (FIG. 1B) or single PTMs on adjacent nucleosomes.

To quickly and efficiently identify a linker that would allow contact of each PCD with a H3K27me3 ligand, we used an in vitro expression and ELISA procedure to test four Pc₂TF variants. Different lengths and physical characteristics were explored by using flexible glycine-serine linkers and rigid alpha-helical linkers. Glycine and serine, amino acids with small side chains, have been used in a wide range protein engineering applications to build linker peptides that have minimal interference with the function of tethered proteins. However, as was demonstrated by mutagenesis of a rigid linker in the bivalent protein BPTF, added flexibility can destabilize protein-histone interactions. Rigid linkers might perform better by stabilizing the distance between PCDs to support interactions with neighboring K27me3 moieties. The Pc₂TF constructs included two tandem copies of the PCD separated by one of four linkers: flexible SEQ. ID. NO. 1: (GGGGS)₄, long flexible SEQ. ID. NO. 2: (GGGGS)₁₆, rigid SEQ. ID. NO. 3: (EAAAR)₄, and long rigid SEQ. ID. NO. 4: (EAAAR)₁₆. Based on a simplified layout of the interacting components (PCDs and a nucleosome carrying two H3K27me3 modifications) (FIG. 1B), we predicted that 20 amino acids would provide sufficient length for adjacent PCDs to bind simultaneously. The 80-amino-acid linkers were used to determine the impact of increased spacing between PCDs.

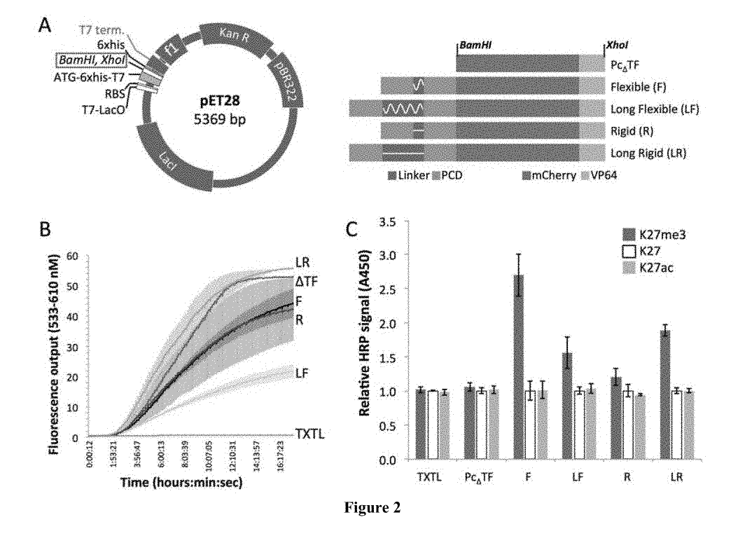

To expedite the prototyping stage, we used a cell-free expression system. Pc₂TF variants and a control protein with no binding domain (PcATF) were expressed from a pET28 vector (FIG. 2A) in TXTL solution. Real-time detection of mCherry fluorescence in a Roche thermal cycler confirmed expression of recombinant proteins. For ELISAs, biotinylated histone peptides were immobilized on a neutravidin-coated 96 well plate. HRP-conjugated anti-mCherry was used for immunodetection of bound fusion proteins. Significantly higher HRP signal was detected compared to background (unmodified K27 and K27ac) for variants that contained the flexible SEQ. ID. NO. 1 (GGGGS)₄, long flexible SEQ. ID. NO. 2 (GGGGS)₁₆, and long rigid SEQ. ID. NO. 3 (EAAAR)₁₆ linkers (FIG. 2C). The implications of these results are discussed in depth the Conclusions. Assuming that HRP signal is proportional to Pc-fusion molecules bound, the flexible SEQ. ID. NO. 1 (GGGGS)₄ linker conferred the strongest avidity in this assay. Therefore, we used this variant in subsequent experiments to determine the impact of bivalency on the activity of synthetic, histone-binding effectors.

### Bivalency Strengthens the Avidity of the Pc-fusion for H3K27me3.

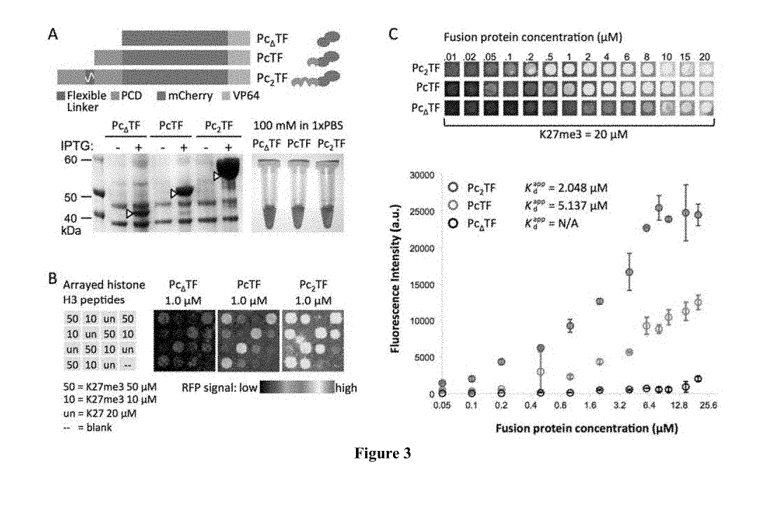

To compare known concentrations of Pc-fusion proteins in subsequent experiments, we over-expressed and purified recombinant proteins from E. coli. Denaturing polyacrylamide electrophoresis (PAGE) of lysates from IPTG-treated and untreated E. coli confirmed inducible production of the proteins at roughly the expected sizes: 37, 44, and 52 kilo Daltons for PC_{Δ}TF, PcTF, and Pc₂TF respectively (FIG. 3A). Nickel-NTA column-purified proteins were soluble in 1× phosphate buffered saline (PBS). The visible red hue under white light, which is typical of the mCherry protein, indicated proper protein folding (FIG. 3A).

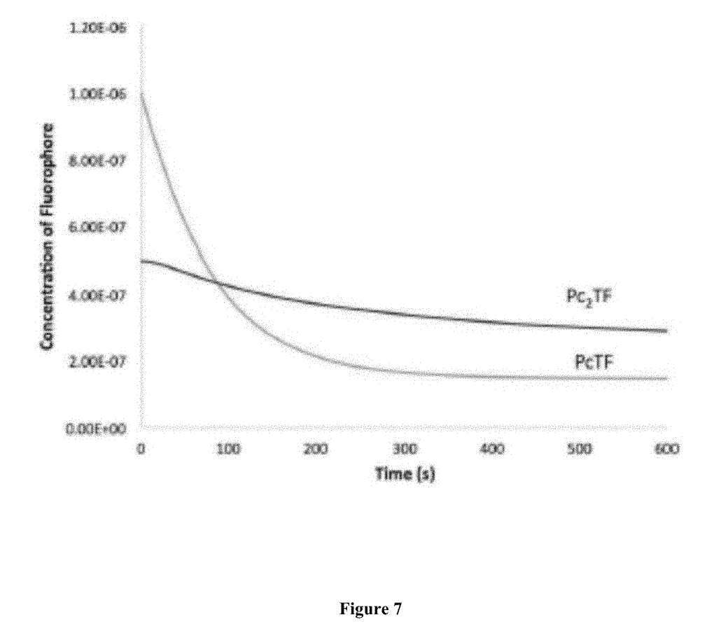

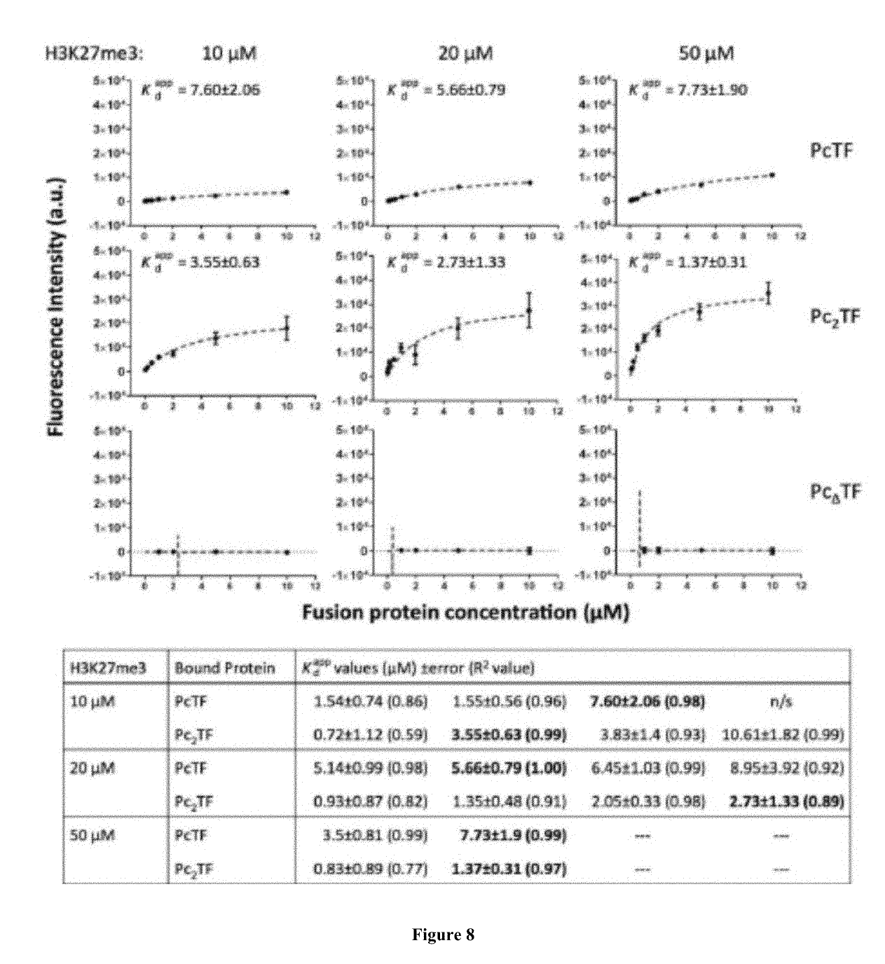

To determine impact of the additional PCD domain on PcTF avidity, we exposed tethered histone peptides to varying concentrations of soluble PcTF and Pc₂TF. Liquid phase ligand (H3K27me3 peptide) binding assays (fluorescence polarization, FP) reported by other groups have determined affinities of N-terminal PCD motifs from the Drosophila Pc protein (residues 1-90, K_{d}=5.0±1 μM¹³) and mammalian CBX8 protein (mouse residues 1-62, K_{d}=165±20 μM¹²; human residues 8-61, K>500 μM). The amino acid sequence of the PCD in our fusion proteins (human CBX8 residues 1-62) is identical to the mouse ortholog. To acquire data that is relevant to the full-length fusion proteins (295 to 445 residues) that we had previously tested as gene regulators in cancer cell lines we used a histone peptide microspot array. We used a mathematical model to predict relative RFP signal levels after Pc-fusion binding and subsequent washing of the microarray. PcTF has a higher mCherry: PCD ratio than Pc₂TF (1:1 vs. 1:2). Therefore, PcTF should show higher relative RFP signal when all targets (H3K27me3 peptides) within a microspot are saturated by PCD binding (FIG. 7). Assuming that bivalency supports an additive increase in avidity, a higher fraction of Pc₂TF molecules should remain bound at the microspot during washing, resulting in higher total RFP signal. Results from a test array were consistent with this prediction (FIG. 3B). We tested concentrations of the recombinant proteins over two orders of magnitude (0.2-20 μM) to determine the apparent dissociation constant (K_{d}^{app}) of each protein for 10, 20 and 50 μM of tethered H3K27me3 ligand (FIG. 8). We detected no interaction with unmodified histone H3 peptides and very little signal above background for the PcΔTF negative control. The K_{d}^{app} of monovalent PcTF was 5.14-8.95 μM for four independent trials (FIG. 3C and S2). The micromolar k_{d}^{app} values are comparable to K_{d} values from the aforementioned FP experiments, although the wash steps in microspot assay may bias K_{d}^{app} towards the off kinetics of the binding process. We conclude that PCD retains its intrinsic affinity for H3K27me3 as an N-terminal motif within a fusion protein.

Overall, analysis of the microspot array data suggest that at 10 and 20 μM of H3K27me3 the K_{d}^{app} of Pc₂TF is roughly 2-fold smaller than PcTF (FIG. 8). Assuming that the second PCD fold (PCD2) maintains its intrinsic affinity, PCD2 should approximately double the overall association rate for Pc₂TF since there is twice the chance of a PCD-H3K27me3 collision. Avidity is related to the inverse of the equilibrium constant, and the equilibrium constant is proportional to the ratio of association rate over dissociation rate. Thus, the effect we observed is most likely due to increasing the association rate and/or decreasing the dissociation rate, which would decrease the k_{d}^{app} value (compared to PcTF) roughly 2-fold.

### Bivalent Pc₂TF Shows Cooperative, on-target Binding with Solid Phase Target Ligands.

Next, we investigated the binding properties of the mono- and bivalent PCD proteins over a range of target ligand densities to approximate dynamic distributions of H3K27me3 that may occur in chromatin. We assumed that random distribution within each mixture would decrease the spacing between H3K27me3 targets as their concentration was increased. We applied dilutions of the target ligand (0-100% H3K27me3 mixed with unmodified H3K27, 1000 nM final concentration) to ELISA wells and exposed the immobilized ligands to the highest concentration of fusion proteins that produced minimal background signal in preliminary ELISA trials (0.1 μM). At 0-15% H3K27me3, HRP signal for PcTF or Pc₂TF was not significantly greater than the negative control fusion protein (Pc_{Δ}TF). In this range, the number of fixed H3K27me3 ligands may not have captured enough fusion proteins to yield detectable signal after washing. At 20% -30% and higher, the HRP signal from the PcTF and Pc₂TF wells increased with H3K27me3 concentration.

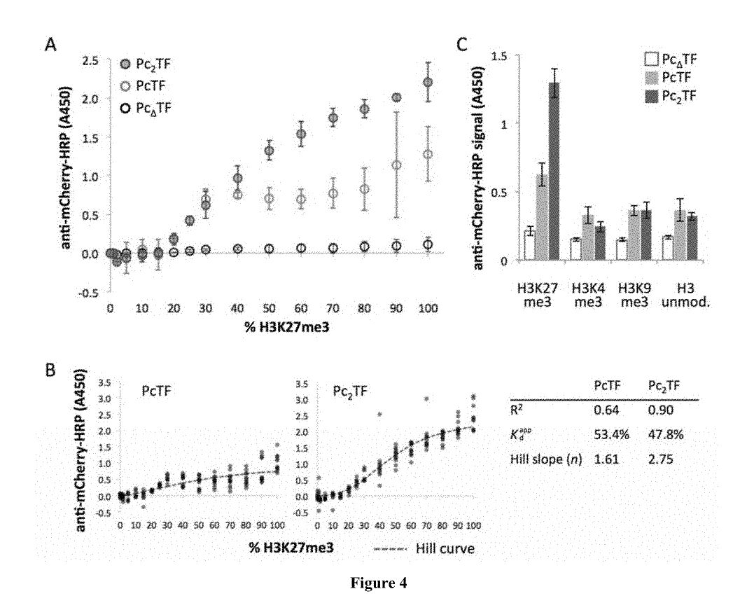

A Hill slope of 2.75 from the nonlinear regression (R²=0.90) for Pc₂TF (FIG. 4B) indicates that binding scales non-linearly with the concentration of its ligand. It is difficult to fit a Hill curve to the data for PcTF (R²=0.64) because the increase in HRP signal is interrupted by a plateau at 30% -80% H3K27me3. The cause of the plateau is unclear, however it is possible that the increase observed above 80% is due to the binding avidity between PcTF and H3K27me3 being exceeded at these concentrations. Overall, we can conclude from the ELISA data that Pc₂TF binding is cooperative.

To investigate ligand selectivity, 50 nM of purified PcTF, Pc₂TF, or Pc_{Δ}TF was tested for interaction with histone peptides that were trimethylated at different lysine residues. PcTF and Pc₂TF showed significant binding with H3K27me3 peptides compared to the control protein Pc_{Δ}TF (FIG. 4C). HRP signal from the H3K27me3 wells was significantly higher than what appears to be non-specific binding with unmodified H3. This was not the case for off-target ligands H3K4me3 and H3K9me3, suggesting that the Pc-fusions can discriminate between the different methyl marks. No significant increase in HRP signal in the off-target wells was observed for Pc₂TF, suggesting that target preference was not lost as avidity was enhanced. One might expect cross-reactivity with H3K9me3 since this PTM appears within a similar motif (ARKS) as H3K27me3. Others have reported that in vitro, chromodomain peptides from different orthologues (CBX1-8) have varying preferences for the two histone modifications. CBX8, the PCD used for PcTF in our work, has shown low affinity for H3K27me3 and none for H3K9me3, which is consistent with our results.

The results from the assays with purified proteins led us to ask, what is the biological consequence of increased binding in living cells where the physical distribution of H3K27me3 is much different. In the cellular chromatin environment, H3K27me3 can occur in cis on the radial surface of a single nucleosome (FIG. 1B), in trans where DNA bending brings the H3 tails of neighboring nucleosomes close together, or sparsely distributed across many nucleosomes. Furthermore, H3K27me3 marks in living cells are dynamic. The enzyme EZH½ adds methyl groups to H3K27 and the enzymes KDM6A (UTX) and KDM6B (JMJD3) remove these marks. We set out to compare Pc₂TF to PcTF in a cellular milieu.

### Bivalent Pc₂TF Activates a Target Gene in a Partially-Silenced State.

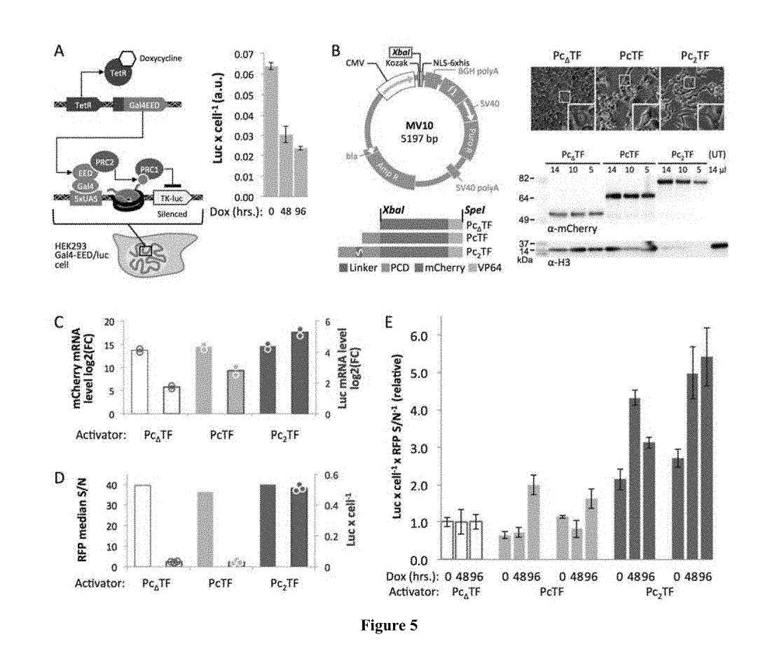

Previously, we demonstrated that PcTF activated a reporter gene near ectopic H3K27me3 in HEK293 cells. Here, we determined the biological significance of PCD bivalency by comparing the gene-regulation activities of Pc₂TF and PcTF at the same reporter. Doxycycline (dox)-mediated induction Gal4-EED in HEK293 Gal4-EED/luc cells leads to accumulation of H3K27me3 at and silencing of a chromosomally integrated Tk-luciferase transgene (FIG. 5A). Tk-luciferase repression reaches steady state at 96 hours, and repression is maintained by epigenetic inheritance after loss of Gal4-EED.

We transfected dox-treated cells (96 hours) with the Pc_{Δ}TF negative control, PcTF, or Pc₂TF cloned into a mammalian expression vector (FIG. 5B). Fluorescence microscopy and Western blots confirmed nuclear localization and expression of full length proteins. Using reverse transcription followed by quantitative PCR (RT-qPCR), we detected higher luciferase transcript levels in Pc₂TF and PcTF-expressing cells compared to Pc_{Δ}TF (FIG. 5C). These results indicate that bivalent Pc₂TF is a stronger activator than PcTF. Luciferase (luc) activity levels detected by an enzymatic assay (FIG. 5D) and normalized to RFP signal-to-noise ratios from flow cytometry corroborated the RT-qPCR results; Pc₂TF stimulated greater luc expression than PcTF. We did not detect significantly higher luc activity for PcTF-expressing cells versus the negative control in this trial (FIG. 5D), but did so in additional experiments (FIG. 5E).

Expression of fusion regulators in cells that were treated with dox for 0, 48, and 96 hours showed that Pc₂TF had roughly twice the activity as PcTF (96 hours), and that Pc₂TF activated Tk-luciferase without prior dox-induced silencing (FIG. 5E). The latter result can be explained by an intermediate, partially silenced level of Tk-luciferase expression compared to fully active Tk-luciferase in a “Luc14” parental cell line that lacks the Gal4-EED gene, as previously observed. H3K27me3 was detected via ChIP-qPCR near the luciferase promoter (Tk) in uninduced Gal4-EED/luc cells at significantly higher levels than in Luc14 cells. Dox treatment resulted in a further decrease in Tk-luciferase expression and a significant increase in H3K27me3 accumulation. In the experiments reported here, basal Tk-luciferase expression (FIG. 5A) agrees with independent experiments from our prior study (0.02-0.07 luciferase activity per cell, a.u.). The uninduced state may have low levels of H3K27me3 at nucleosomes near the reporter gene in all cells, or high levels of H3K27me3 at the reporter gene in a small proportion of cells in the population. In contrast to Pc₂TF, monovalent PcTF only activated Tk-luciferase after silenced chromatin had been induced for 96 hours. These results suggest that Pc₂TF is more tolerant of low levels of H3K27me3 in cellular chromatin.

In the embodiments and examples disclosed herein, there are demonstrations of modular, synthetic multivalency of a chromatin-derived, histone-binding protein with gene-regulating activity. We have demonstrated for the first time the use of a monovalent synthetic effector to activate chromatin-silenced genes in live cells. Natural bivalent chromatin proteins that recognize two histone post translational modifications at once suggest a broader design space for synthetic chromatin effectors. Our application of bivalency to design a synthetic fusion protein produced two important advances for engineering synthetic chromatin effectors. First, we determined that synthetic linkers allow tethered histone PTM-binding peptides to function within the context of a fusion protein in vitro and in live cells. Second, we have established that doubling the valency with tandem PCDs strengthens avidity and increases gene regulation activity by at least 2-fold.

Here, we demonstrated that different synthetic linkers allow tethered histone PTM-binding peptides to bind in vitro to varying degrees. We observed weaker binding for the longer flexible linker (80 amino acids) compared to the shorter linker in our ELISA experiment. This result is likely due to lower production of the long flexible linker variant in TXTL. Given that both variants showed binding above background, SEQ. ID. NO. 5 (GGGGS)-repeat number may not significantly affect bivalent PCD engagement with H3K27me3 in vitro. For the rigid linker-tethered PCDs, only the longer length (80 amino acids) appeared to support binding. Assuming that this variant protein was properly folded, lack of binding over background for the shorter SEQ. ID. NO. 6 (EAAAR)-repeat variant could be caused by suboptimal rotation, i.e., in trans instead of in cis, of the second PCD away from the 2-D binding surface in the ELISA well. This mechanism was demonstrated with mutated alpha-helical linkers in bivalent BPTF and with tandem Zinc Finger DNA binding domains. In the context of cellular chromatin where looping and folding occurs, H3K27me3 would not necessarily be constrained to one face of the Pc₂TF protein. Valuable insights and perhaps greater Pc₂TF performance might be acquired by exploring additional linker variants in cells as well as in vitro.

We have established that tandem PCDs strengthen avidity for H3K27me3 in a cooperative manner in vitro and increase gene regulation activity in live cells by at least 2-fold compared to a monovalent PCD. The wide distribution of multivalency within bromodomain family and other effector proteins suggests that multivalent engagement has an important, evolutionarily-conserved biological role. Multivalency appears to largely be represented by cell-cycle and gene-activating effectors. Relatively few multivalent proteins that recognize silencing marks have been studied in biophysical detail. Examples include the chromodomains of the Arabidopsis protein CMT3 and the mammalian protein HP1β; as bivalent homodimers, these proteins show enhanced interaction with their respective ligands H3K9meK27me and H3K9me3. Pc₂TF is novel in its composition of histone-binding motifs: adjacent, identical Polycomb chromodomains within a single peptide. Therefore, its activity in vitro and in cells provides new insights into the recognition of histone marks by effector proteins.

In the context of cellular chromatin, Pc₂TF appears to be active at the target gene prior to full repression (FIG. 5E, 0 hours dox), whereas detectable activity of monovalent PcTF required a prolonged period of induced repression at the target (FIG. 5E, 96 hours dox). Our previous ChIP mapping data confirm that compared to the fully active and fully silenced states, intermediate levels of H3K27me3 appear at Tk-luciferase (on average) without the addition of doxycycline. It is likely that in the pre-treated state, leaky Gal4-EED expression causes a few cells in the population have one or two H3K27me3 marks at a nucleosome near the Tk promoter. Stronger avidity, supported by the additional PCD module, may increase the likelihood of an activation event at the target in this small population of cells. This idea is consistent with the behavior of synthetic zinc finger-based DNA-binding regulators, where stronger affinity of the regulator for its DNA target is associated with stronger gene activation. Similar behavior can also be observed for multivalent receptor-binding peptides, which bind with high avidity and specificity to a small number of receptor-positive cells.

Further engineering efforts to achieve greater, nonlinear enhancement of PcTF/Pc₂TF may require changes within the PCD binding motif. The hydrophobic interaction between the methylammonium cage and the methyl-lysine moiety (Fig. 1A) depends upon proper positioning of PCD residues that appear discontinuously in the primary sequence; this positioning requires specific intramolecular contacts of peptide residues within the PCD fold. Reverse engineering and de novo design of a new binding pocket through randomization of sequences would likely yield many non-functional proteins. K27-adjacent interactions that contribute to interactions with the histone tail could be leveraged to enhance affinity. However, increasing the stability by introducing additional hydrogen bonding could overwhelm the hydrophobic, K27me3-specific interaction, and allow PCD to recognize unmodified tails or off-target modifications. Trade-offs between affinity and specificity pose formidable challenges to enhancing PCD affinity. Therefore, the most practical strategy for identifying alternative PCDs is to leverage H3K27me3-specific orthologs and paralogs from various species. It will be important to determine cross-reactivity with different histone modifications since certain CBX PCD peptides have been shown to bind H3K9me3.

Multivalent engagement of combinatorial histone marks has recently become a key line of evidence to support the controversial histone code hypothesis. Rationally-designed synthetic multivalency will advance this important area of research by exploring functions beyond the limits of pre-existing natural multivalent proteins. Furthermore, engineered chromatin effectors provide a practical tool to support artificial regulation of gene expression states through direct engagement with highly-conserved components of chromatin, i.e. histone tails and their modifications. Therapeutic, synthetic gene regulators that leverage this mechanism could help circumvent the shortcomings of epigenetic inhibitors, which target chromatin enzymes that can gain drug-resistant mutations. This demonstrates that synthetic biology is a powerful tool for fundamental investigations of chromatin biology and epigenetic engineering.

### Exemplary Methods

Plasmid Constructs for TXTL and Bacterial Expression. Constructs (FIG. 2A, FIG. 3A) were assembled as BioBrick compatible fragments in vector V0120. Fragments were PCR-amplified with Phusion polymerase using primers 1-6 (Table 1, below) and a protocol adapted from New England Biolabs Phusion High Fidelity DNA Polymerase (98° C. 0:45, [98° C. 0:10, 67° C. 0:20, 72° C. 0:45]×25, final extension of 5:00), column purified (Qiagen PCR Cleanup Kit), and double-digested with BamHI and Xhol (New England BioLabs). BamHI/Xhol-digested inserts and 50-75 ng BamHI/XhoI linearized pET28(+) vector were ligated at a 3:1 molar ratio in a 20 μL reaction as described in the New England BioLabs (NEB) protocol for T4 ligase (M0202). 5 μL of each ligation was incubated with 50 μL Turbo competent DH5-alpha E. coli (NEB) on ice for 5 minutes, transferred to 45° C. for 45 seconds, then to ice for 5 minutes, and allowed to recover in 350 μL SOC at 37° C. with shaking for 30 minutes. Pelleted cells were resuspended in 50 μL SOC, plated on LB agar (50 ug/mL kanamycin), and grown at 37° C. overnight. Colony PCR was performed to identify positive ligation results using primers 6 and 7 (Table 1) and the GoTaq Promega protocol. Plasmids were cloned, extracted (Sigma GeneElute Plasmid Miniprep Kit), and Sanger sequenced for verification prior to protein expression in cell-free TXTL or in E. coli. Annotated sequences for all pET28 constructs are available online at Benchling-Hayneslab: Synthetic Chromatin Actuators 2.0 (benchling.com/hayneslab/f_/rmSYkAAU-synthetic-chromatin-actuators-2-0).

TABLE 1
Primers 1-6 were used for addition of restriction sites to pre-assembled
constructs via Phusion PCR to build the bacterial/TXTL expression plasmids.
Primers 7 and 8 were used for verification by Sanger sequencing. Primers 9
and 10 were used to amplify inserts for the MV10 vector. Non-binding
overhangs are shown.

| Template(s) | Primer Name | Primer Sequence (5′ ...) |
|---|---|---|
| PcTF_V0120 | 1. PcTF.pET28.For.2 | SEQ. ID. NO. 7: |
| | | atgtcaGGATCCATGGAGCTTTCAGCGGTG |
| PcTF_V0120 | 2. PcTF.pET28.Rev | SEQ. ID. NO. 8: |
| | | GCGCTTTTTCTTGGGCTCGAGCAACATGTCCA |
| | | AGTCG |
| PcTF_pET28 | 3. mcVP64.For | SEQ. ID. NO. 9: |
| | | aatgcctGGATCCATGGTGAGCAAGGGCGAGGA |
| PcTF_pET28 | 4. mcVP64.Rev | SEQ. ID. NO. 10: ATCTCAGTGGTGGTGGTGG |
| PcTF_V0120 | 5. DD.pET28.For | SEQ. ID. NO. 11: |
| | | cataacaGGATCCGCGGCCGCATCTAGAATG |
| PcTF_V0120 | 6. DD.pET28.Rev | SEQ. ID. NO. 12: |
| | | acttgggCTCGAGGCGGCCGCTACTAGT |
| pET28 | 7. T7.For | SEQ. ID. NO. 13: |
| | | TAATACGACTCACTATAGGGGAATTG |
| pET28 | 8. T7.Rev | SEQ. ID. NO. 14: GCTAGTTATTGCTCAGCGG |
| PcTF_V0120, | 9. Biobrick For | SEQ. ID. NO. 15: |
| Pc₂TF_V0120 | | TCACTGACTGACTGACTGCGTCTCAA |
| PcTF_V0120, | 9. Biobrick For | SEQ. ID. NO. 16: |
| Pc₂TF_V0120 | | TTCCAGTCAGTCAGTCAGTCGTCTCTTG |

TXTL: Cell-free Expression. TXTL reactions were set up with the following conditions as previously described: 9 μL lysate, 10 nM final template vector, 0.5 nM σ70-T7 RNA pol vector to a total of 12 μL. A Roche Lightcycler 480 was used to detect mCherry fluorescence with the following: protocol: 30° C. for 10 minutes, bring to 29° C. for 1 second, scan 533-610 nm, repeat 96 times (total 16 hrs).

E. coli Expression and Purification of Proteins. All selection media contained 50 μg/mL kanamycin. Pc_{Δ}TF, PcTF, and Pc₂TF in pET28 were transformed into Rosetta 2pLys DE3 cells and plated on LB agar and grown at 37° C. overnight. The next day, a single colony from each was used to inoculate 50 mL LB and grown overnight at 37° C. at 300 RPM. The next day, 1 liter of LB in a baffled Erlenmeyer flask was inoculated to an OD₆₀₀ of 0.1. The cultures were grown to an OD₆₀₀=0.6, induced with IPTG (1 mM final concentration) and allowed to express Pc₆₆ TF and PcTF at 37° C. for 5 hours with shaking (220 RPM). Pc₂TF-expression was carried out overnight at room temperature with shaking (220 RPM) to aid solubility of the protein. Cell disruption and protein purification are described in detail below. Purification of recombinant protein from E. coli. also is described below.

Enzyme-linked immunosorbent assays (ELISAs). All steps were carried out at room temperature except specifically noted, and all incubations and washes were agitated at 800 RPM on an Eppendorf Thermomixer R. Clear bottom plates (Greiner bio-one #655101) were coated in 50 μL of 20 ng/μL neutravidin in PBS pH. 8.0 overnight at 4° C. The plates were washed the next day 3× with 200 μL 0.2% PBS-Tween (PBST) with 5 minutes of shaking at 800 RPM between washes. The plate was blocked for 30 min at 800 RPM at room temperature with 200 uL 5% BSA in 0.2% PBST followed by 3× washes of 200 μL 0.2% PBST for 5 minutes each at 800 RPM. 50 μL of 1μM biotinylated peptides (Anaspec: H3 (21-44), H3K4me3 (1-21), H3K9me3 (1-21), H3K27me3 (21-44), or H3K27Ac (21-43)) in 0.2% PBST) were incubated at room temperature for 1 hour at 800 RPM, followed by 3× washes of 200 μL 0.2% PBST for 5 minutes at 800 RPM. The plate was blocked with 200 μL 5% skim milk in 0.2% PBST (room temperature, 800 RPM, 30 min.). 1.5 μL TxTL (FIG. 2), or 50 μL of 0.1 μM (FIG. 3A) or 0.05 nM (FIG. 3C) purified proteins in 50 μL 5% skim milk in PBST were incubated in each well for 1 hour (room temperature, 800 RPM). The wells were washed 3x with 200 μL 5% skim milk in PBST with 5 minutes of 800 RPM shaking. After adding 100 μL of 1:3000 chicken polyclonal anti-mCherry (Novus Biologicals #NBP2-25158) in 5% nonfat milk in 0.2% PBST, wells were incubated for 1 hour, followed by 3× of 200 uL 5% skim milk in 0.2% PBST for 5 min each (room temperature, 800 RPM). After adding 100 uL of 1:3000 Rabbit anti-chicken-HRP (RCYHRP) Genetel 0.5 mg/mL) in 5% skim milk in 0.2% PBST wells were incubated for 30 minutes (room temperature, 800 RPM). The plate was washed 5× with 200μL 0.2% PBST for three minutes each at 800 RPM. The plate was incubated with 100 μL of 1-step Ultra TMB-ELISA (Thermo-Fisher #34029) for 15 minutes while protected from light. Reactions were stopped with 100 μL 2.0 M sulfuric acid, incubated for 2 min, and read at 450 nm. Each plate contained four technical replicates per H3K27me3 concentration, per fusion protein. Two ELISA plates (trials) were run for each of two purified protein samples per construct. One trial failed to show significant signal over background (for all recombinant proteins) and was omitted from the final analysis. In FIG. 4 “anti-mCherry-HRP (A450)”=HRP signal-mean HRP signal for 0% H3K27me3. The Microsoft Excel Solver tool was used

$$\frac{1}{{( \frac{{K}_{d}^{app}}{[ L ]} )}^{n} + 1}$$

to fit the Hill equation to the data by minimizing the sum of the squared errors between the equation and data (varying K_{d}^{app} and n). R² was calculated as 1−(SS_{reg}/SSₜₒₜ), where total sum of squares SSₜₒₜ=Σᵢ(yᵢ−y̅)² and regression sum of squares SS_{reg}=Σᵢ(fᵢ−y̅)²

Peptide Microspot Arrays. APTES functionalized glass slides were coated with 200 μL of 1:1 (v/v) 40 mg/mL BS3 crosslinking solution and 1 mg/mL neutravidin with a cover slide (Thermo Scientific, #651-2-5251) and incubated overnight at 4° C. The next day, the cover slide was removed and the slide was rinsed 3× with 0.2% PBST for 5 minutes each. Slides were deactivated by incubation with Na₂CO₃/NaHCO₃ buffer pH 9.4 for 30 minutes. The slides were quickly rinsed with ddH₂O, and centrifuged to dry at 1200 RPM for 2 minutes. Slides were printed with biotinylated peptides (Anaspec) at concentrations of 10, 20, or 50 μM in 20% glycerol and PBS with a pin-printer (spot to spot distance=600 μm) and incubated at room temperature for 1 hr. The slide was rinsed with ddH₂O as described above and blocked with superblock for 1 hr at room temperature. Proteins were diluted in superblock and incubated on the slide for 1 hr at room temperature. The slides were rinsed with 0.2% PBST for 3 minutes each followed by quick rinsing with ddH₂O 3× and centrifuged dry (as described). Red fluorescent protein (mCherry) signal was detected at 50% gain and 50% intensity on a PowerScanner at 635 nm and 535 nm, 10 μm resolution. Slides were also scanned at 75%-75% and 100%-100% to obtain a suitable signal to noise ratio. Arraypro software was used to quantify the median intensity values for each spot and background levels. Graphpad Prism software was used to fit the binding saturation nonlinear regression equation y=(Bₘₐₓ*X)/(K_{d}^{app}+X) to the data, where Bₘₐₓ is the highest binding value, and X is the concentration of protein.

Plasmid Constructs for Mammalian Expression. MV10 was constructed from pcDNA3.1(+) (Invitrogen) with the following modifications. The CMV promoter was removed via SpeI digestion and T4 ligase recircularization. A dsDNA fragment that encodes Kozak (ribosome binding site), XbaI, a nuclear localization sequence, 6x histidine, and a stop codon SEQ. ID. NO. 17(5′-cccgccgccaccatggagtctagacccaagaaaaagcgcaaggtacaccatcaccaccatcacgcgtaaagctgag) with SpeI overhangs at both ends (ctag/t) was inserted at XbaI. CMV (SpeI/XbaI fragment) was reintroduced upstream of Kozak at SpeI. Proper orientation of inserts was confirmed by Sanger sequencing. Constructs PcTF and Pc₂TF (FIG. 5B) were PCR-amplified (Phusion) with primers 9 and 10 (Table 1), double-digested with XbaI and SpeI, and column-purified (Qiagen PCR Purification, 28104). Construct Pc_{Δ}TF (FIG. 5B) was double-digested with XbaI and SpeI (Thermo Fisher FastDigest) and isolated by electrophoresis and gel purification. XbaI/SpeI fragments and 25 ng XbaI-linearized, dephosphorylated MV10 vector were ligated at a 2:1 molar ratio in a 10 μL reaction as described in the Roche protocol for Rapid DNA Ligation (11635379001 Roche), using 1.0 μL NEB T4 ligase instead of the supplied enzyme. All 10 μL of each ligation was incubated with 50 μL Turbo competent DH5-alpha E. coli (New England Biolabs) on ice for 5 min, transferred to 45° C. for 45 seconds, then to ice for 5 min. Cells were plated directly on pre-warmed LB agar (100 ug/mL ampicillin) without recovery and grown at 37° C. overnight. Plasmid DNA was prepared (Sigma GeneElute Plasmid Miniprep Kit) from 5 mL cultures inoculated with single colonies. Forward orientation of the inserts was determined by XbaI and PstI double-digestion of prepped plasmids and Sanger sequencing. Annotated sequences for all MV10 constructs are available online at Benchling-Hayneslab: Synthetic Chromatin Actuators 2.0 (available at benchling.com/hayneslab/f_/rmSYkAAU-synthetic-chromatin-actuators-2-0).

Cell Culture and Transfection. HEK293 Gal4-EED/luc cells were grown in Dulbecco's modified Eagle's medium (DMEM) supplemented with 10% tetracycline-free fetal bovine serum and 1% penicillin and streptomycin at 37° C. in a humidified CO₂ incubator. Silencing of the reporter gene (Tk-luciferase) was induced by supplementing the media with 1 μg/mL of dox for 48 or 96 hours. For wash-out of doxycycline (to allow depletion of Gal4-EED), growth medium was removed and replaced with dox-minus medium supplemented with 0.5 μg/mL puromycin to select for the transgenic anti-Gal4-EED shRNA, and grown for 5 days. Prior to transfection, dox treated or untreated cells were plated in 12-well culture dishes at 40% confluency (˜1.0E5 cells per well) in 2 mL pen/strep-free growth medium. Transient transfections were carried out by adding 100 μL of DNA/Lipofectamine complexes to each well: 1 μg pDNA or ddH₂O for mock transfections (10 μL), 3μL Lipofectamine LTX (Invitrogen), 87 μL Opti-MEM. 48 hours after transfection, cellular mCherry (580/610 excitation/emission) was imaged in culture dishes on a Nikon Eclipse Ti wide field inverted fluorescent microscope (MEA53100) at 200× magnification (eyepiece=10×; objective=CFI S Plan Fluor ELWD 20×, numerical aperture=0.45), 25° C., without oil immersion, and with either phase contrast or an mCherry filter set (TE2000 cube, excitation FF01-562/40-25, emission FF01-641/75-25). Images from each channel were acquired with a digital monochrome camera (Coolsnap ES2 12 bit, 20 MHz) and overlaid using NIS-Elements software. For downstream assays (RT-qPCR, Western blots, and flow cytometry), the growth medium was removed, semi-adherent cells were gently collected with 1× PBS washes, pelleted (200 g, room temperature, 5 min) and resuspended in 1× PBS. Six replicate samples (wells) were pooled for assays in FIGS. 5B, C, and D.

RT-qPCR. Preparation of total RNA, cDNA synthesis, and qPCR were performed as previously described using ˜1.0E6 HEK293 Gal4-EED/luc cells that were pelleted (500 g, room temperature, 5 min) and lysed with 500 μL TRIzol (Thermo Fisher #15596026). DNA/LNA oligos for qPCR were: mCherry-forward SEQ. ID. NO. 18 (5′-cctgaagggcgagatcaag), reverse SEQ. ID. NO. 19 (5′-ttgacctcagcgtcgtagtg), LNA probe #41 (Millipore Sigma #04688007001); luciferase-forward SEQ. ID. NO. 20 (5′-caggtcttcccgacgatg), reverse SEQ. ID. NO. 21 (5′-gtattccgtgctccaaaac), LNA probe #70 (Millipore Sigma #04688937001); GAPDH (reference)-Roche human G6PD assay (Millipore Sigma #5046246001). Mean Crossing point (Cp), the first peak of d²y/dx² (fluorescence over cycle number), was calculated by the Roche LightCycler 480 software for three replicate wells per unique reaction. For each biological replicate (one transfection per fusion protein) two replicate cDNA synthesis reactions (from one RNA prep) were completed. Expression level was calculated as delta Cp=2̂[Cp_{reference}−Cp]. “mRNA level log2(FC)”=delta Cp transfected cells/delta Cp mock.

Western blots. Total protein was prepared from roughly 250,000 cells. Sample preparation, polyacrylamide gel electrophoresis (PAGE), and membrane blotting are described in detail in Supporting Information. Immunostaining was carried out with the following: blocking buffer-5% nonfat dry milk in 1× PBST (1x PBS, 0.1% Tween-20); primary 1-chicken polyclonal anti-mCherry, 1:2000 (Novus Biologicals #NBP2-25158); secondary 1-HRP-conjugated rabbit anti-chicken, 1:2000 (Millipore Sigma #AP162P); primary 2-anti-histone H3, 1:1000 (Abcam #ab1791); secondary 2- HRP-conjugated goat anti-rabbit, 1:2000 (Cell Signalling Technology #7074). Immunostaining was performed at 4° C. overnight (primary) or at room temperature for 1 hour (secondary) with nutation in a Parafilm pouch. Immunostained blots were washed 4×10 min in 1× PBST, with orbital shaking at room temperature. HRP signal was detected using the SuperSignal West Femto substrate kit (Thermo Fisher #34095) and a PXi4 imager (Syngene) with GeneSys software.

Imaging and flow cytometry. Cells were passed through a 35 μm nylon strainer (EMS #64750-25). Red fluorescent signal from mCherry was detected on a BD Accuri C6 flow cytometer (675 nm LP filter) using CFlow Plus software. Data were further analyzed using FlowJo 10.0. One run (˜10,000 live cells, gated by forward and side scatter) was completed per sample. “RFP median signal/noise (S/N)”=median RFP signal from live RFP-positive cells/median RFP noise from live untransfected cells.

Luciferase Assays. Cell counts (per 100 μL) were determined by flow cytometry (BD Accuri C6). 100 μL of cells or 1× PBS (blank) were incubated with 100 μL of complete luciferase assay reagent as described in the protocol for the Biotium Firefly Luciferase Assay Kit (89138-960) and in previous work in Corning and Costar 96-well Cell Culture Plates, opaque, white (Corning 3789A). Chemiluminescence was detected using a Synergy H1 Multi-Mode Reader (Biotek). Replicates included three samples (100 μL each) taken from a single population of transfected cells. “Luc ×cell⁻¹(a.u.)” =[Sample Luciferase signal]—1×PBS blank signal/[cell count ×(100 μL/20 μL)]. For fusion protein-expressing cells, normalization was performed by dividing Luc ×cell⁻¹ by the RFP median signal/noise value (from flow cytometry).

Purification of recombinant protein from E. coli. IPTG-induced cells were pelleted by centrifugation at 4,000 RCF for 10 min, resuspended in 30 mL of purification buffer (10% glycerol, 250 mM NaCl, 50 mM Na2PO4, pH 8.0), and frozen at −80° C. overnight. Disruption of thawed cells (on ice) was performed by sonication with a QSONICA instrument (model Q500) at 50% power: (1 second on, 2 seconds off)×6000 cycles. After addition of imidazole (10 mM final concentration), insoluble material was pelleted at 16,000 RPM for 30 min at 4° C. in a Beckman Coulter Avante J-E Rotor JA-17. Purification columns were prepared by washing 2.5 mL of Ni-NTA Agarose (Qiagen #30210) with 10 mL of ddH20 on a 50 mL polyprep column, then equilibrated with 15 mL purification buffer plus 10 mM imidazole. Soluble fractions of cell lysates were loaded onto a plugged column, vortexed briefly to homogenize resin and supernatant, and incubated with rotation at 4° C. for 2 h. The cap and bottom plug were removed to empty the unbound fraction, and flow-through was applied back to the column once. Protein-bound resin was washed with 10 mL binding buffer plus 10 mM imidazole, followed by 5 mL binding buffer plus 20 mM imidazole and 5 mL binding buffer plus 50 mM imidazole. Resin was incubated with 1 mL of binding buffer plus 250 mM imidazole for 10 min in a plugged column before elution. Elution was repeated with binding buffer plus 500 mM, and then binding buffer plus 1.0 M imidazole. Proteins were concentrated and buffer-exchanged into PBS using a 15 mL 30,000 kDa centrifugal filter and repeated washes of PBS followed by centrifugation at 4000 RCF for 10 min, and stored at 4° C. in a final volume of about 1 mL. Concentration of protein was determined using a denaturing polyacrylamide gel with bovine serum albumin (BSA) standards.

Western blot detailed protocol. Roughly 250,000 cells were pelleted at 300 g for 5 min, lysed by resuspension in 500 μL Mammalian Cell PE LB (G-Biosciences #786-180) plus 5 μL 100× ProteaseArrest (G-Biosciences #786-108), and vortexed for 2 min. Insoluble debris was pelleted at 16,000 xg, 4° C., 5 min and discarded. Samples for denaturing polyacrylamide gel electrophoresis (PAGE) were prepared by heating 15 μL lysate plus 4 μL NuPAGE® LDS Sample Buffer 4×(Thermo Fisher #NP0007) and 1 μL 1M DL-Dithiothreitol (DTT, Millipore Sigma #D0632-1G) at 100° C. for 5 min. Samples (cooled to room temperature) and a pre-stained protein standard (10 μL, Thermo Fisher #10748010) were electrophoresed at 120 V in a 4-12% Bis-Tris gel (Thermo Fisher #NP0322BOX) with MOPS-SDS buffer [50 mM MOPS, 50 mM Tris Base, 0.1% SDS, 1 mM EDTA, pH 7.7] diluted 1:20 in an XCell SureLock vertical chamber (Invitrogen #EI0001). Proteins were transferred onto a nitrocellulose membrane (Bio-Rad #1704158) via semi-dry transfer in a Transblot Turbo system (Bio-Rad #1704150). Complete transfer was verified by staining the membrane with 1×Ponceau-S (G-Biosciences #786-576).

The following claims are not to be limited to the embodiments and examples disclosed herein. All references herein are hereby incorporated by reference in their entirety.

What is claimed is:

1. A histone-binding fusion-protein composition, comprising a modified polycomb chromodomain (PCD) motif.

2. The composition of claim 1, wherein said modified polycomb chromodomain (PCD) motif comprises two tandem copies of the H3K27me3-binding PCD at the N-terminus.

3. The composition of claim 1, wherein said modified polycomb chromodomain (PCD) motif comprises two tandem copies of the H3K27me3-binding PCD at the N-terminus and separated by a linker.

4. The composition of claim 3, wherein said linker is selected from the group consisting of SEQ. ID. NO. 1: (GGGGS)₄, SEQ. ID. NO. 2: (GGGGS)₁₆, SEQ. ID. NO. 3: (EAAAR)₄, and SEQ. ID. NO. 4: (EAAAR)₁₆.

5. The composition of claim 3, wherein said linker is SEQ. ID. NO. 1: (GGGGS)₄.

6. The composition of claim 1, wherein said modified polycomb chromodomain (PCD) motif strengthens avidity and increases gene regulation activity by at least 2-fold compared with an unmodified PCD.

7. The composition of claim 2, wherein said modified polycomb chromodomain (PCD) motif strengthens avidity and increases gene regulation activity by at least 2-fold compared with an unmodified PCD.

8. The composition of claim 3, wherein said modified polycomb chromodomain (PCD) motif strengthens avidity and increases gene regulation activity by at least 2-fold compared with an unmodified PCD.

9. The composition of claim 2, wherein said histone-binding fusion-protein binds H3K27me3 on multiple histone proteins.

10. A method for multivalent engagement of one or more histone proteins by a transcriptional regulator, comprising contacting said one or more histone proteins with a histone-binding fusion-protein composition having a modified polycomb chromodomain (PCD) motif.

11. The method of claim 10, wherein said modified polycomb chromodomain (PCD) motif comprises two tandem copies of the H3K27me3-binding PCD at the N-terminus.

12. The method of claim 10, wherein said modified polycomb chromodomain (PCD) motif comprises two tandem copies of the H3K27me3-binding PCD at the N-terminus and separated by a linker.

13. The method of claim 12, wherein said linker is selected from the group consisting of SEQ. ID. NO. 1: (GGGGS)₄, SEQ. ID. NO. 2: (GGGGS)₁₆, SEQ. ID. NO. 3: (EAAAR)₄, and SEQ. ID. NO. 4: (EAAAR)₁₆.

14. The method of claim 12, wherein said linker is SEQ. ID. NO. 1: (GGGGS)₄.

15. A method for tuning the activity of a synthetic histone-binding transcriptional regulator, comprising the step of modifying a polycomb chromodomain (PCD) motif of said synthetic histone-binding transcriptional regulator.

16. The method of claim 15, wherein said tuning comprises modifying the PCD motif to include two tandem copies of the H3K27me3-binding PCD at the N-terminus.

17. The method of claim 15, wherein said tuning comprises modifying the PCD motif to include two tandem copies of the H3K27me3-binding PCD at the N-terminus and separated by a linker.

18. The method of claim 17, wherein said linker is selected from the group consisting of SEQ. ID. NO. 1: (GGGGS)₄, SEQ. ID. NO. 2: (GGGGS)₁₆, SEQ. ID. NO. 3: (EAAAR)₄, and SEQ. ID. NO. 4: (EAAAR)₁₆.

19. The method of claim 17, wherein said linker is SEQ. ID. NO. 1: (GGGGS)₄.

20. The method of claim 19, wherein said modified PCD motif strengthens avidity and increases gene regulation activity by at least 2-fold compared with an unmodified PCD.

21. The method of claim 19, wherein said synthetic histone-binding transcriptional regulator binds H3K27me3 on multiple histone proteins.


SEQUENCE LISTING (21 sequences)

SEQ ID NO: 1 - protein, 20 aa, Artificial Sequence
  Synthetic Construct
GGGGSGGGGSGGGGSGGGGS

SEQ ID NO: 2 - protein, 80 aa, Artificial Sequence
  Synthetic Construct
GGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 3 - protein, 20 aa, Artificial Sequence
  Synthetic Construct
EAAAREAAAREAAAREAAAR

SEQ ID NO: 4 - protein, 80 aa, Artificial Sequence
  Synthetic Construct
EAAAREAAAREAAAREAAAREAAAREAAAREAAAREAAAREAAAREAAAREAAAREAAAREAAAREAAAREAAAREAAAR

SEQ ID NO: 5 - protein, 5 aa, Artificial Sequence
  Synthetic Construct
GGGGS

SEQ ID NO: 6 - protein, 5 aa, Artificial Sequence
  Synthetic Construct
EAAAR

SEQ ID NO: 7 - DNA, 30 bp, Artificial Sequence
  Synthetic Construct
atgtcaggatccatggagctttcagcggtg

SEQ ID NO: 8 - DNA, 37 bp, Artificial Sequence
  Synthetic Construct
gcgctttttcttgggctcgagcaacatgtccaagtcg

SEQ ID NO: 9 - DNA, 33 bp, Artificial Sequence
  Synthetic Construct
aatgcctggatccatggtgagcaagggcgagga

SEQ ID NO: 10 - DNA, 19 bp, Artificial Sequence
  Synthetic Construct
atctcagtggtggtggtgg

SEQ ID NO: 11 - DNA, 31 bp, Artificial Sequence
  Synthetic Construct
cataacaggatccgcggccgcatctagaatg

SEQ ID NO: 12 - DNA, 28 bp, Artificial Sequence
  Synthetic Construct
acttgggctcgaggcggccgctactagt

SEQ ID NO: 13 - DNA, 26 bp, Artificial Sequence
  Synthetic Construct
taatacgactcactataggggaattg

SEQ ID NO: 14 - DNA, 19 bp, Artificial Sequence
  Synthetic Construct
gctagttattgctcagcgg

SEQ ID NO: 15 - DNA, 26 bp, Artificial Sequence
  Synthetic Construct
tcactgactgactgactgcgtctcaa

SEQ ID NO: 16 - DNA, 28 bp, Artificial Sequence
  Synthetic Construct
ttccagtcagtcagtcagtcgtctcttg

SEQ ID NO: 17 - DNA, 76 bp, Artificial Sequence
  Synthetic Construct
cccgccgccaccatggagtctagacccaagaaaaagcgcaaggtacaccatcaccaccatcacgcgtaaagctgag

SEQ ID NO: 18 - DNA, 19 bp, Artificial Sequence
  Synthetic Construct
cctgaagggcgagatcaag

SEQ ID NO: 19 - DNA, 20 bp, Artificial Sequence
  Synthetic Construct
ttgacctcagcgtcgtagtg

SEQ ID NO: 20 - DNA, 18 bp, Artificial Sequence
  Synthetic Construct
caggtcttcccgacgatg

SEQ ID NO: 21 - DNA, 20 bp, Artificial Sequence
  Synthetic Construct
gtctttccgtgctccaaaac